(19) United States
(12) Patent Application Publication
(10) Pub. No.: US 2005/0040757 A1
(43) Pub. Date: Feb. 24, 2005

(54) Light-emitting organic oligomer compositions

(75) Inventors: Shaw H. Chen, Penfield, NY (US); Yanhou Geng, Changchan (CN)

Correspondence Address: OLIFF & BERRIDGE, PLC, P.O. BOX 19928, ALEXANDRIA, VA, 22320, US

(73) Assignee: UNIVERSITY OF ROCHESTER, Rochester, NY (US)

(21) Appl. No.: 10/898,223

(22) Filed: Jul. 26, 2004

Related U.S. Application Data
(60) Provisional application No. 60/489,905, filed on Jul. 25, 2003.

(51) Int. Cl. C07C 43/20; H01J 1/62; H01J 63/04

(52) U.S. Cl. 313/504; 568/633; 546/37

(57) ABSTRACT

Oligomer compositions include conjugated oligomers having fused fluorene residues flanking an aromatic group. Synthetic methods include reacting fused fluorene residues with aromatic compounds.

This non-provisional application claims the benefit of U.S. Provisional Application No. 60/489,905, filed Jul. 25, 2002. The entire disclosure of the provisional application is hereby incorporated by reference herein in its entirety.

This invention was made with United States government support from the Army Research Office under Agreement No. DAAD19-01-1-0676, and the National Science Foundation under Agreement No. CTS-0204827. The United States government may have certain rights in this invention.

### BACKGROUND OF THE INVENTION

This invention is directed to monodisperse electroluminescent conjugated oligomer compositions. This invention is further directed to methods for preparing such compositions.

Organic materials with extended π-conjugation hold promise for electronic and photonic applications, such as thin-film transistors, light emitting diodes, photovoltaics, lasers and sensors. Good processability, high luminance efficiencies and good chemical, thermal and photo stability are important properties in commercial applications. The use of conjugated polymers as macromolecular luminescent materials has been actively studied. Such polymers have been used in the fabrication of various photonic devices.

Luminescent polymers offer the ability to coat substrates efficiently by any of a variety of common solution processing techniques such as spin coating. In addition, the optical and electrical properties of such polymers can be fine-tuned in various ways by varying their structure. A wide variety of polymers, including poly(p-phenylenevinylene) (PPV), polythiophene (PT), poly(p-phenylene) (PPP), polyfluorene (PF) and their derivatives, have been investigated as emissive materials. See, e.g., Bemius et al., “Progress from Light-Emitting Polymers,” Adv. Mater., 12:1737 (2000). Various improvements have been made with respect to ease of synthesis and processability, control of charge carrier balance, overall device efficiency and lifetime and, to some extent, emissive wavelength tunability.

Polymers are generally rigid in nature, which enhances their susceptibility to inter-chain aggregation leading to undesired emission color shift. Long polymer molecules are also prone to entanglements and defects (e.g., bends or kinks). The manufacturing processes for various types of polymer molecules are often very complicated and polymers are subject to malformed polynuclear structures and branching during synthesis. The presence of such impurities and defects can result in materials having decreased luminescence efficiency. Synthetic polymers can also suffer from high polydispersity and low glass transition temperatures, properties that hinder the formation of high quality monodomain films. Finally, many polymer compounds exhibit problems with solubility and stability.

In contrast to conjugated polymers, monodisperse conjugated oligomers are characterized by a well-defined and uniform molecular structure (i.e., chain length and sequence). In the absence of chain entanglements or defects, relatively short and uniform oligomer chains are more conducive to the formation of monodomain films. Because impurities may result in decreased luminescence through exciton quenching, oligomers may also benefit from superior chemical purity.

However, oligomers are generally more prone to crystallization than polymers, resulting in polycrystalline films that scatter light and limit charge injection and transport. A critical issue for their use is the ability to accomplish uniaxial molecular alignment across a large area through molecular self-assembly and nematic mesomorphism. In this regard, materials capable of glass transition while resisting crystallization could form glassy films without grain boundaries. Such materials would be well suited for device applications.

Few conjugated oligomers have been reported to exhibit thermotropic nematic mesomorphism. Recently, a series of monodisperse oligofluorenes have been reported as an example of glassy-nematic conjugated oligomers. See, e.g., Y. Geng et al., “Monodisperse Oligofluorenes Forming Glassy-Nematic Films for Polarized Blue Emission,” Chem. Mater., 15:542 (2003). Such blue light emitting oligomers exhibit a T_{g} of about 150° C. and a T_{c} beyond 375° C., and form monodomain glassy nematic films via spin coating, thermal annealing and cooling to room temperature without encountering crystallization.

To this end, π-conjugated nematic liquid crystals that emit the full color spectrum and are capable of preserving molecular alignment in the solid state upon cooling while bypassing crystallization are highly desirable.

### SUMMARY OF THE INVENTION

In various exemplary embodiments, monodisperse oligomer compositions including conjugated oligomers and synthetic methods for obtaining such compositions are provided. In further exemplary embodiments, glassy-nematic films prepared from monodisperse oligomer compositions according to this invention are provided. In still further exemplary embodiments, organic light emitting devices including glassy-nematic films according to this invention are provided.

In various exemplary embodiments, conjugated oligomers included in monodisperse oligomer compositions according to this invention are soluble, highly processable and chemically tunable to generate polarized color emissions covering the full color range. In various exemplary embodiments, conjugated oligomers included in monodisperse oligomer compositions according to this invention exhibit thermotropic nematic mesomorphism for electronic and photonic applications such as light-emitting diodes (LEDs), and flat panel displays.

In various exemplary embodiments, conjugated oligomers included in monodisperse oligomer compositions according to this invention are characterized by a uniform molecular structure of rod-like molecules having a large aspect ratio and an emission transition dipole parallel to a long molecular axis. Such oligomers resist crystallization and are conducive to formation of monodomain glassy-nematic films across a large area. In this regard, materials capable of glass transition while resisting crystallization and are thus better suited for device application, such as for example, organic light-emitting diodes (OLEDs) and flat panel displays.

In various exemplary embodiments, oligomer compositions according to this invention include conjugated oligomers having fused fluorene residues. In various exemplary embodiments, oligomer compositions according to this invention include conjugated oligimers including fused fluorene residues flanking an aromatic group. In various exemplary embodiments, oligomer compositions according to this invention include conjugated oligomers including fluorene residues and fused fluorene residues flanking an aromatic group.

In various exemplary embodiments, methods for synthesizing oligomer compositions according to this invention include obtaining fluorene residues and fused fluorene residues. In various exemplary embodiments, fluorene residues and fused fluorene residues are combined to prepare combined fluorene/fused fluorene residues. In various exemplary embodiments, combined fluorene/fused fluorene residues are combined with aromatic compounds to prepare conjugated oligomers according to this invention.

### DETAILED DESCRIPTION OF EXEMPLARY EMBODIMENTS

Embodiments of this invention include light-emitting organic oligomer compositions comprising at least one light-emitting oligomer.

Embodiments of oligomer compositions according to this invention are monodisperse. Monodisperse compositions have an absence of significant chain length distribution (i.e., substantially all molecules have the same number of monomer units or residues) or molecular weight. As used in this application, monodisperse compositions include those compositions having a polydispersity factor of less than about 3. In embodiments, oligomer compositions according to this invention have a polydispersity factor of from about 1 to about 2. In embodiments, oligomer compositions according to this invention have a polydispersity factor of from about 1 to about 1.5. In some embodiments, oligomer compositions according to this invention have a polydispersity factor of about 1.

The substantial uniformity in shape and size of constituent oligomers in embodiments of oligomer compositions according to this invention results in improved chemical purity over known compositions. Furthermore, because embodiments of compositions according to this invention do not have a significantly distributed molecular length within the composition, the compositions do not suffer from the effects of varying properties throughout the composition. Embodiments of oligomer compositions according to this invention can be easily processed into uniform thin films having temporal stability of emissive color.

Embodiments of oligomer compositions according to this invention exhibit a high degree of uniaxial alignment. In embodiments, oligomer compositions according to this invention exhibit an orientational order parameter (S) of at least about 0.4. In other embodiments, oligomer compositions according to this invention exhibit an orientational order parameter (S) of from about 0.4 to about 1.0. In embodiments, oligomer compositions according to this invention exhibit an orientational order parameter (S) of from about 0.5 to about 1.0. In embodiments, oligomer compositions according to this invention exhibit an orientational order parameter (S) of from about 0.7 to about 1.0.

Embodiments of oligomer compositions according to this invention include conjugated oligomers having fused fluorene residues. As used herein, “fused fluorene” refers to indenofluorenes, such as indenofluorene and bisindenofluorene. The number and position of such fused fluorene residues within a conjugated oligomer are not particularly limited. In various exemplary embodiments, oligomer compositions according to this invention include conjugated oligomers including two or more fused fluorene residues. In various exemplary embodiments, oligomer compositions according to this invention include conjugated oligomers including fused fluorene residues flanking an aromatic group. In various exemplary embodiments, oligomer compositions according to this invention include conjugated oligomers including fluorene residues and fused fluorene residues flanking an aromatic group.

Embodiments of oligomer compositions according to this invention include conjugated oligomers, including a central segment flanked by segments of fluorene units with aliphatic pendants. In various exemplary embodiments, one or more of the flanking fluorene units are fused fluorene units. Exemplary embodiments of conjugated oligomers included in the oligomer compositions according to this invention are described by the following general formulas (I) and (II): 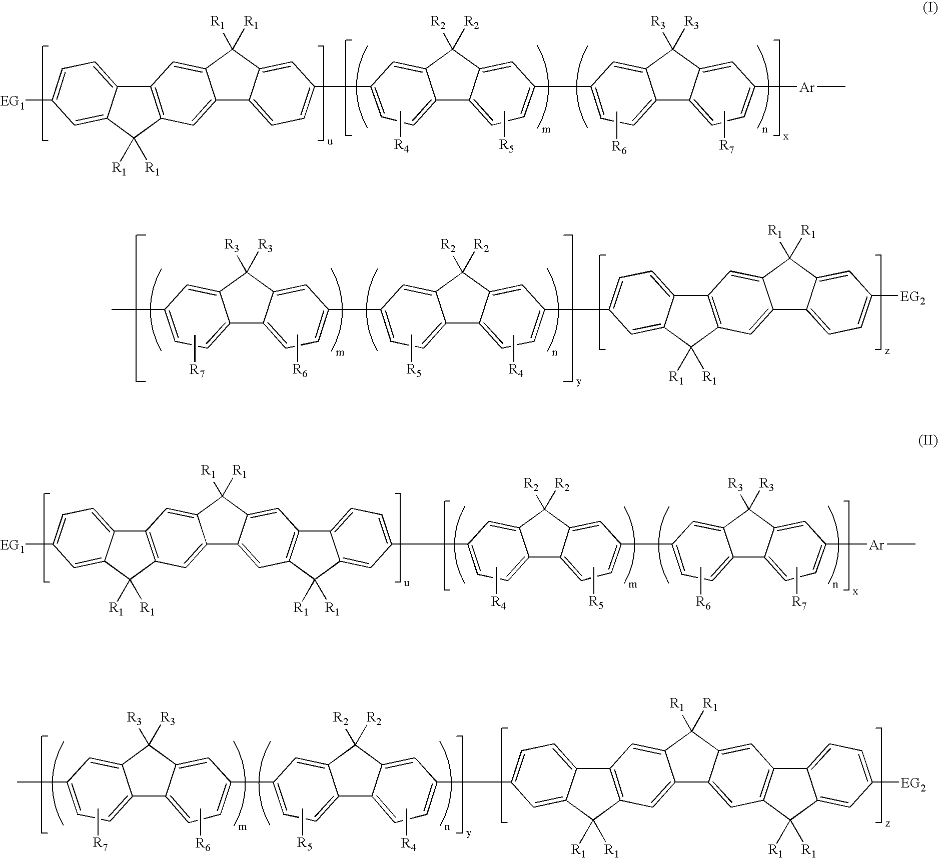
wherein:

R₁, R₂, R₃, R₄, R₅, R₆, R₇ each independently represent C_{q} branched or straight chain aliphatic groups, preferably C_{q}H_{2q+1} or (CH₂CH₂O)_{q}CH₃, racemic, chiral or achiral;

q represents an integer of from about 1 to about 40, preferably from 1 to 20;

m, n, u, x, y, z each independently represent an integer of from 0 to about 10, and u+z+(m+n)(x+y) is an integer of from about 2 to about 40, preferably from about 2 to about 20;

EG₁, EG₂ each independently represent end groups, preferably selected from the group consisting of H, a linear or branched alkyl group, and an unsubstituted or substituted aryl group; and

Ar represents a group selected from the group consisting of:

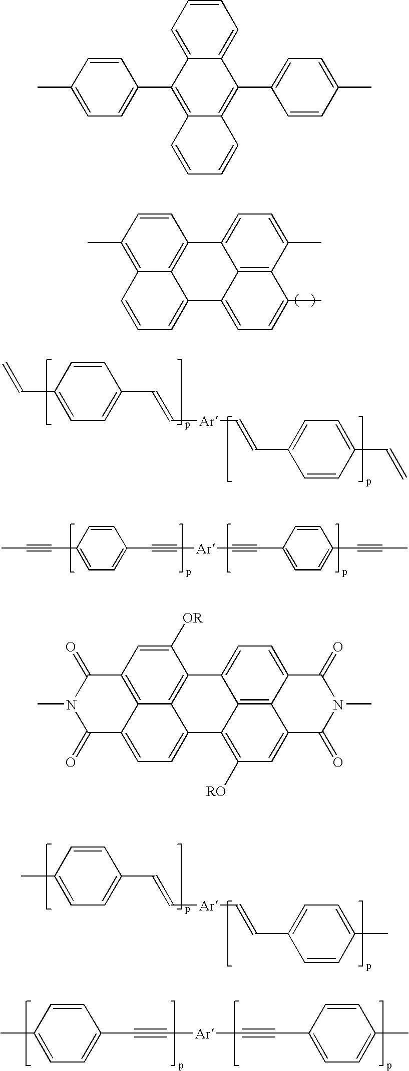

where p=0 to 10, preferably from 0 to 5.

Ar′ represents a group selected from the group consisting of:

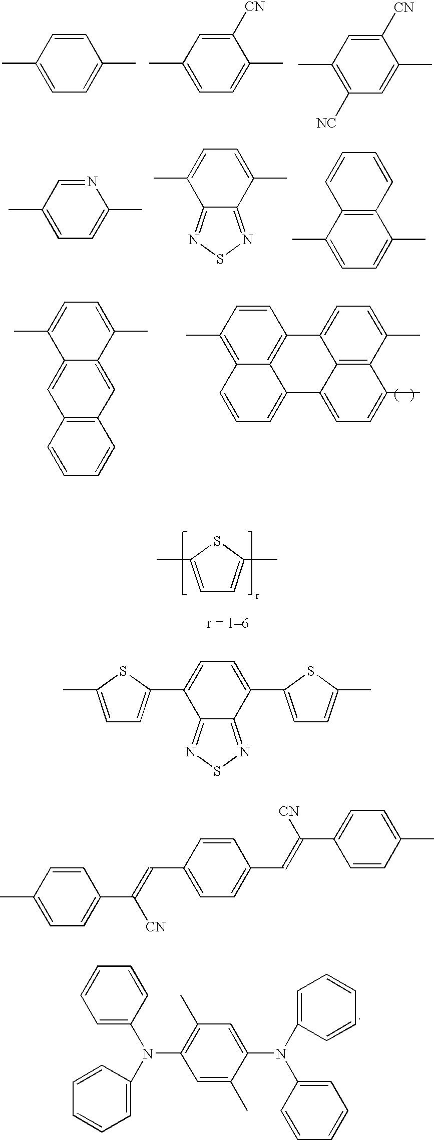

In various exemplary embodiments, the fused fluorene residues include indenofluorenes and bisindenofluorenes. In various exemplary embodiments, fluorene residues in conjugated oligomers of oligomer compositions according to this invention include pendant aliphatic groups. In some such embodiments, the aliphatic groups are branched or straight alkyl groups. In various exemplary embodiments, pendant aliphatic groups ensure the solubility of an oligomer in a solvent.

Embodiments of the present invention include compositions containing one or more oligomers according to one or both of formulas (I) and (II), in any combination, as desired for a particular application of a composition.

In various exemplary embodiments, oligomer compositions according to this invention include conjugated oligomers having uniform molecular structure with emissive colors that are chemically tunable.

In various exemplary embodiments, oligomer compositions according to this invention include conjugated oligomers having a large aspect ratio and an emission transition dipole parallel to a long molecular axis of central segments responsible for light emission.

In various exemplary embodiments, oligomer compositions according to this invention include conjugated oligomers that can be formed into monodomain glassy-nematic films across a large area. For example, oligomer compositions according to this invention can be formed into monodomain glassy-nematic films across areas of sufficient size to form monitors, television screens or components thereof.

In various exemplary embodiments, oligomer compositions according to this invention include conjugated oligomers displaying thermotropic nematic mesomorphism and having glass transition temperatures of from about 90 to 140° C. or from about 97 to 127° C. In various exemplary embodiments, oligomer compositions according to this invention include conjugated oligomers having nematic-to-isotropic transition temperatures of 250° C. or more, 300° C. or more, 350° C. or more.

Embodiments of the present invention include glassy-nematic films prepared from monodisperse oligomer compositions including conjugated oligomers having fused fluorene residues. In various exemplary embodiments, glassy-nematic films can be formed from oligomer compositons including conjugated oligomers according to one or both of formulas (I) and (II). Exemplary films according to this invention can be formed by any technique know to those of ordinary skill in the art. For example, films according to this invention can be formed by spin coating.

In various exemplary embodiments, glassy-nematic films according to this invention have orientation order parameters characterizing molecular alignment of at least about 0.70 or at least about 0.77, as determined by UV-vis absorption dichroism. In various exemplary embodiments, glassy-nematic films according to this invention have an emission dichroic ratio of at least about 8.0 or at least about 9.4, as determined by polarized photoluminescence.

In various exemplary embodiments of glassy-nematic films according to this invention, electron transition dipoles of central segments of constituent oligomers lie largely parallel to long molecular axes of such oligomers. In various exemplary emobodiments, long molecular axes of such oligomers follow a nematic director defined by mechanically buffing the substrate.

Embodiments of the present invention include OLEDs including one or more layers of oligomer films formed from monodisperse oligomer compositions according to this invention.

In various exemplary embodiments, OLEDs according to this invention include glassy-nematic films prepared from monodisperse oligomer compositions including conjugated oligomers having fused fluorene residues. In some such embodiments, OLEDs according to this invention include glassy-nematic films formed from oligomer compositons including conjugated oligomers according to one or both of formulas (I) and (II). In various exemplary embodiments, OLEDs according to this invention emit any color of light with a peak polarization dichroic ratio of at least about 12.0, at least about 14.4 or at least about 18.0. In various exemplary embodiments, OLEDs according to this invention emit any color of light with a luminance yield of at least about 0.40 cd/A, at least about 0.50 cd/A or at least about 0.59 cd/A, at a current of about 20 mA/cm².

In various exemplary embodiments, methods for synthesizing oligomer compositions according to this invention include obtaining fluorene residues and fused fluorene residues. In various exemplary embodiments, the fluorene residues and fused fluorene residues are combined to prepare combined fluorene/fused fluorene residues. In various exemplary embodiments, combined fluorene/fused fluorene residues are combined with an aromatic compound to prepare the conjugated oligomers according to this invention. In various exemplary embodiments, synthetic methods according to this invention result in a monodisperse oligomer composition.

In embodiments, obtained fluorene residues may include a fluorene or two or more linked fluorenes. For example, fluorene residues may include one, two, three, four, five or six linked fluorenes. Fluorenes may be include pendant branched or straight-chain alkyl groups. In various exemplary embodiments, fluorene residues may be functionalized with groups that facilitate joining of fluorene residues to the other constituent portions of conjugated oligomers according to this invention. In various exemplary embodiments, fluorene residues can be functionalized with dioxaborolane groups, trimethylsilyl groups and/or halogen groups (e.g., bromine).

In various exemplary embodiments, obtained fused fluorene residues may include a fused fluorene structure having three or more six-carbon rings. For example, fused fluorene residues may include three, four, five, six, seven or eight six-carbon rings. Fused fluorene residues may include pendant branched or straight-chain alkyl groups. In various exemplary embodiments, fused fluorene residues may be functionalized with groups that facilitate joining fused fluorene residues with to other constituent portions of conjugated oligomers according to this invention. In various exemplary embodiments, fused fluorene residues can be functionalized with dioxaborolane groups, trimethylsilyl groups and/or halogen groups (e.g., bromine).

In various exemplary embodiments of methods for synthesizing oligomer compositions according to this invention, fluorene residues are joined with fused fluorene residues. Fluorene residues can be joined with fused fluorene residues by any suitable method. For example, fluorene residues functionalized with trimethylsilyl groups and dioxaborolane groups can be reacted with fused fluorene residues functionalized with halogen groups. In various exemplary embodiments, fluorene residues and fused fluorene residues can be reacted in the presence of catalysts such as, for example, palladium catalysts. In various exemplary embodiments, such reactions of fluorene residues and fused fluorene residues can result in combined fluorene/fused fluorene residues. In various exemplary embodiments, such combined fluorene/fused fluorene residues can be functionalized with trimethylsilyl groups.

In various exemplary embodiments, combined fluorene/fused fluorene residues are subjected to one or more reactions to prepare the combined fluorene/fused fluorene residues for joining with an aromatic group. In various exemplary embodiments, such preparation can include substituting functional groups of combined fluorene/fused fluorene residues. In various exemplary embodiments, combined fluorene/fused fluorene residues are treated to replace trimethylsilyl groups with halogen groups (e.g., iodine groups). In various exemplary embodiments, such substitution can be conducted by reacting combined fluorene/fused fluorene residues with halogen compounds. For example, combined fluorene/fused fluorene residue functionalized with trimethylsilyl groups can be reacted with halogen compounds such as iodine chloride.

Combined fluorene/fused fluorene residues can then be reacted with aromatic compounds to obtain conjugated oligomers according to this invention. Such reactions to obtain conjugated oligomers according to this invention can be carried out in a single reaction or over several reaction steps. In various exemplary embodiments, combined fluorene/fused fluorene residues can be reacted with aromatic compounds including a desired aromatic unit. For example, combined fluorene/fused fluorene residues functionalized with halogen groups, such as iodine, can be reacted with aromatic compounds such as, for example, 4,7-Bis(p-vinylstyryl)-2,1,3-benzothiadiazole, in the presence of one or more catalysts, such as palladium catalysts. Such reactions yield conjugated oligomers according to this invention, the oligomers including fluorene residues and fused fluorene residues flanking aromatic groups.

In various exemplary embodiments, combined fluorene/fused fluorene residues can be reacted with various compounds to sequentially add components of a desired aromatic group. For example, combined fluorene/fused fluorene residues functionalized with halogen groups, such as iodine, can be reacted with aromatic compounds, such as thiophene compounds. Resulting combined fluorene/fused fluorene residues functionalized with aromatic groups can be further reacted with other aromatic compounds, such as 4,7-dibromo-2,1,3-benzothiadiazole to obtain conjugated oligomers according to this invention, the oligomers including fluorene residues and fused fluorene residues flanking aromatic groups.

Monodisperse oligomer compositions of the present invention can be synthesized by any suitable known or later developed approach or procedure. In various exemplary embodiments, monodisperse oligomer compositions of the present invention can be synthesized using reaction schemes such as reaction schemes (A) and (B), shown and described below.

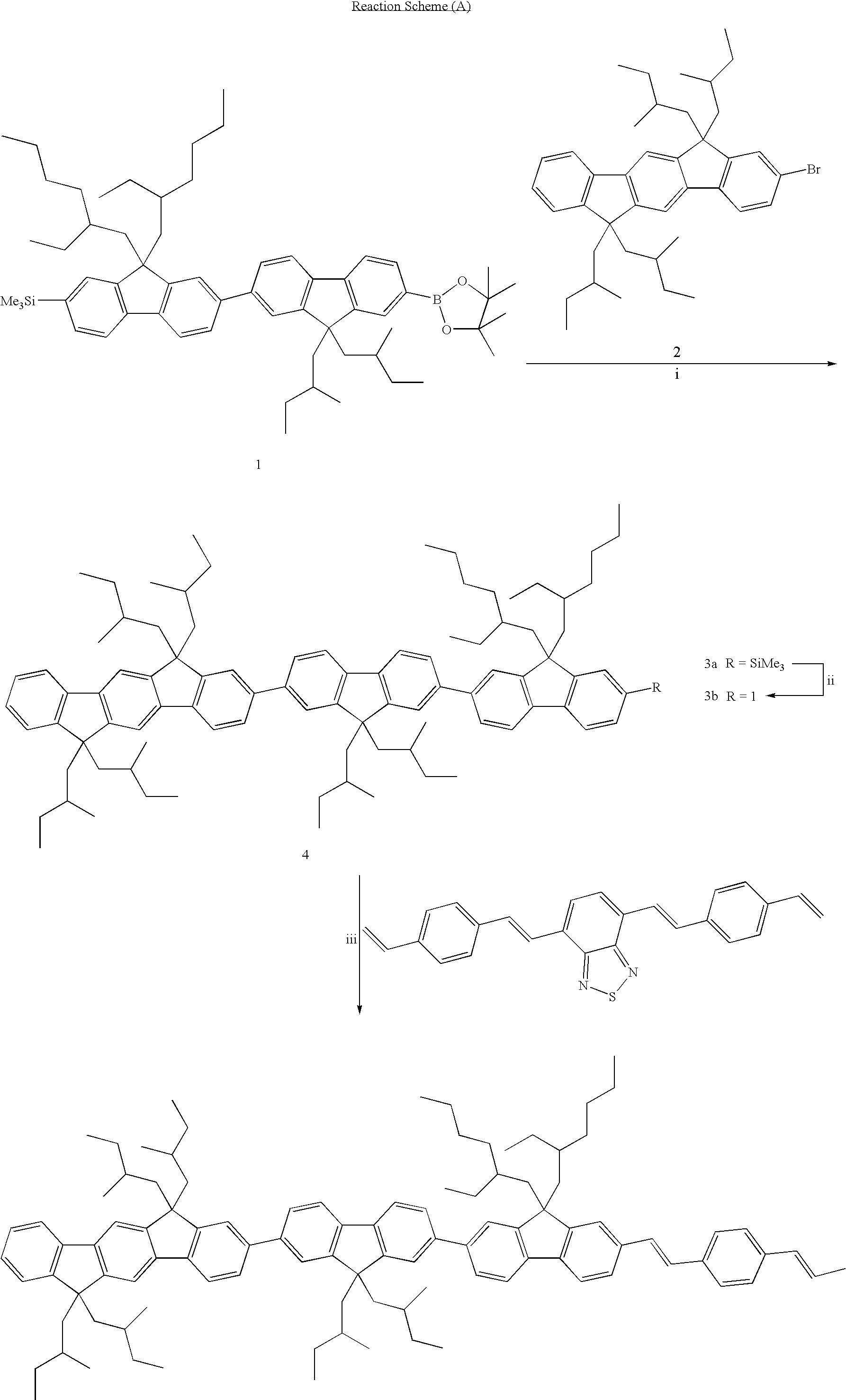

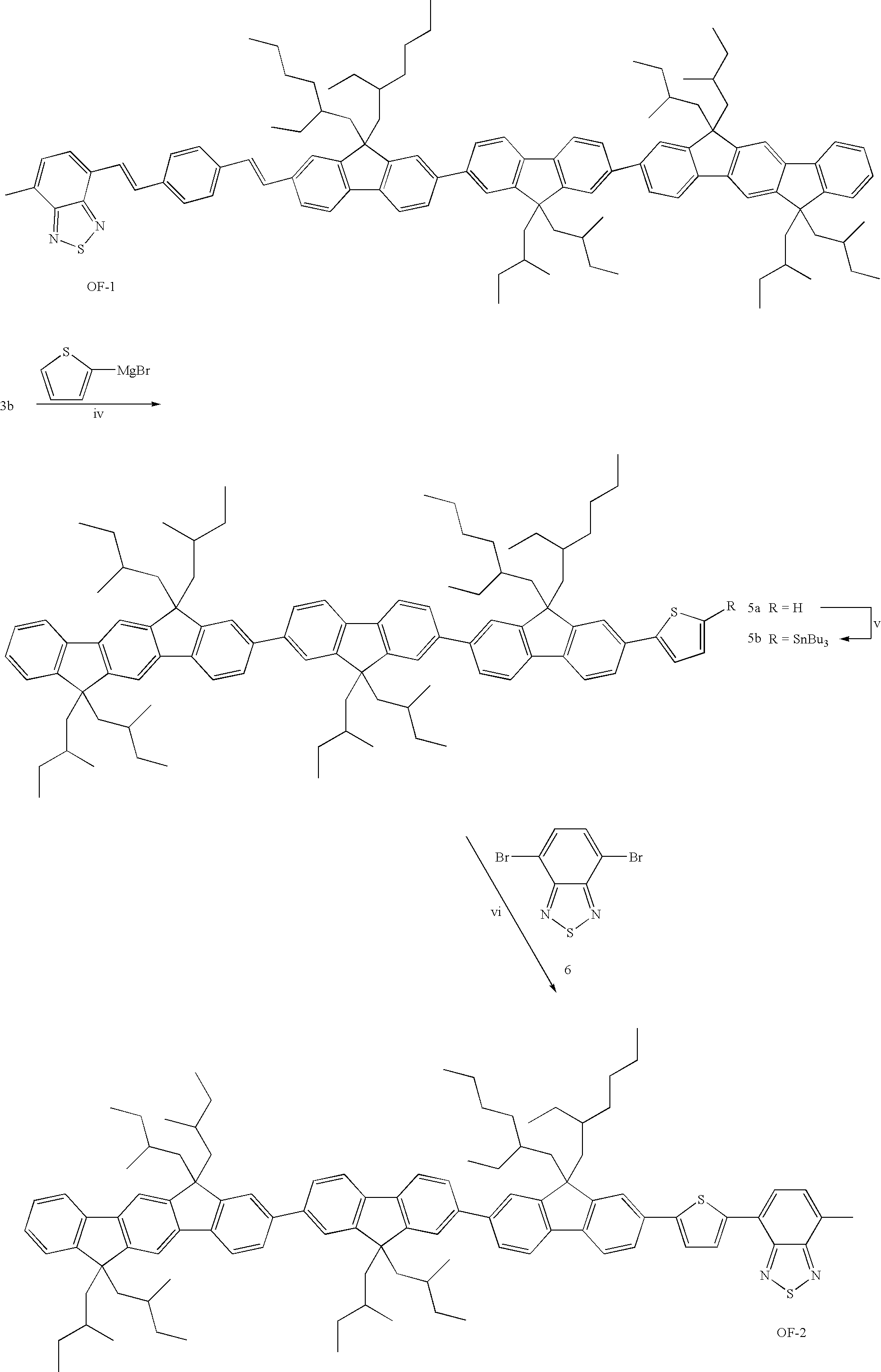

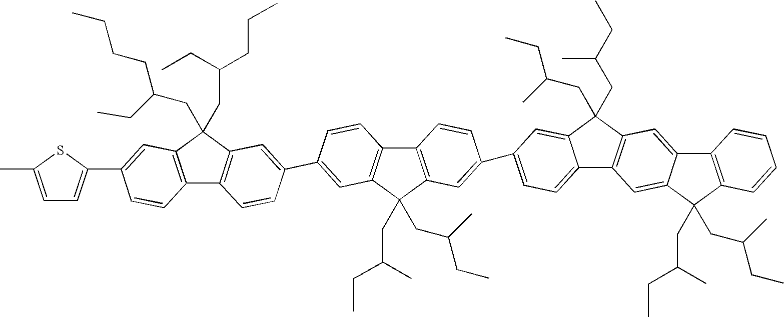

Reaction scheme (A) includes two synthetic pathways, through which monodisperse conjugated oligomer compositions according to this invention can be obtained. In particular, reaction scheme (A) provides the conjugated oligomers 4,7-bis[2-[4-[2-[4-[2-[7′-[6,6,12,12-tetrakis(2-methylbutyl)indenofluoren-2-yl]-9,9-bis(2-ethylhexyl)9′,9′-bis(2-methylbutyl)-2,2′-bifluoren-7-yl]ethenyl]phenyl]ethenyl]phenyl]ethenyl]-2,1,3-benzothiadiazole (OF-1) and 4,7-bis[5-[7′-[6,6,12,12-tetrakis(2-methylbutyl)indenofluoren-2-yl]-9,9-bis(2-ethylhexyl)- 9′,9′-bis(2-methylbutyl)-7,2′-bifluoren-2-yl]-thien-2-yl]-2,1,3-benzothiadiazole (OF-2).

According to the first synthetic pathway in reaction scheme (A), 2-[7′-trimethylsilyl-9,9-bis(2-methylbutyl)-9′,9′-bis(2-ethylhexyl)-7,2′-bifluoren-2-yl]-4,4,5,5-tetramethyl-[1,3,2]dioxaborolane (1) is reacted with 2-bromo-6,6,12,12-tetrakis(2-methylbutyl)indenofluorene (2) in the presence of Pd(PPh₃)₄ in a 2.0 M aqueous solution of Na₂CO₃ at a temperature of 90° C. to obtain 7-[6,6,12,12-tetrakis(2-methylbutyl)indenofluoren-2-yl]-7′-trimethylsilyl-9,9-bis(2-methylbutyl)-9′,9′-bis(2-ethylhexyl)-2,2′-bifluorene (3a).

The obtained 7-[6,6,12,12-tetrakis(2-methylbutyl)indenofluoren-2-yl]-7′-trimethylsilyl-9,9-bis(2-methylbutyl)-9′,9′-bis(2-ethylhexyl)-2,2′-bifluorene (3a) is reacted with IC1 at 0° C. to obtain 7-iodo-7′- [6,6,12,12-tetrakis(2-methylbutyl)indenofluoren-2-yl]-9,9-bis(2-ethylhexyl)- 9′,9′-bis(2-methylbutyl)-2,2′-bifluorene (3b).

The obtained 7-iodo-7′-[6,6,12,12-tetrakis(2-methylbutyl)indenofluoren-2-yl]-9,9-bis(2-ethylhexyl)- 9′,9′-bis(2-methylbutyl)-2,2′-bifluorene (3b) is reacted with 4,7-bis(p-vinylstyryl)-2,1,3-benzothiadiazole (4) in the presence of Pd(OAc)₂, K₂CO₃ and Bu₄NBr at 110° C. to obtain a monodisperse oligomer composition including the conjugated oligomer 4,7-bis[2-[4-[2-[4-[2-[7′-[6,6,12,12-tetrakis(2-methylbutyl)indenofluoren-2-yl]-9,9-bis(2-ethylhexyl)-9′,9′-bis(2-methylbutyl)2,2′-bifluoren-7-yl]ethenyl]phenyl]ethenyl]phenyl]ethenyl]-2,1,3-benzothiadiazole (OF-1).

According to the second synthetic pathway in reaction scheme (A), 7-iodo-7′-[6,6,12,12-tetrakis(2-methylbutyl)indenofluoren-2-yl]-9,9-bis(2-ethylhexyl) 9′,9′-bis(2-methylbutyl)-2,2′-bifluorene (3b) is reacted with 2-thienylmagnesium bromide in the presence of Pd(dppf)Cl₂ at room temperature to obtain 2-[7′-[6,6,12,12-tetrakis(2-methylbutyl)indenofluoren-2-yl]-9,9-bis(2-ethylhexyl)- 9′,9′-bis(2-methylbutyl)-7,2′-bifluoren-2-yl]-thiophene (5a).

The obtained 2-[7′-[6,6,12,12-tetrakis(2-methylbutyl)indenofluoren-2-yl]-9,9-bis(2-ethylhexyl)-9′,9′-bis(2-methylbutyl)-7,2′-bifluoren-2-yl]-thiophene (5a) is reacted with n-BuLi at 0° C. and then Bu₃SnCl at −78° C. The mixture is then returned to room temperature. 2-[7′-[6,6,12,12-Tetrakis(2-methylbutyl)indenofluoren-2-yl]-9,9-bis(2-ethylhexyl)-9′,9′-bis(2-methylbutyl)-7,2′-bifluoren-2-yl]-5-tributylstanyl-thiophene (5b) is thus obtained.

The obtained 2-[7′-[6,6,12,12-tetrakis(2-methylbutyl)indenofluoren-2-yl]-9,9-bis(2-ethylhexyl)-9′,9′-bis(2-methylbutyl)-7,2′-bifluoren-2-yl]-5-tributylstanyl-thiophene (5b) is reacted with 4, 7-dibromo-2,1,3-benzothiadiazole (6) in the presence of Pd(PPh₃)₄ at 100° C. to obtain a monodisperse oligomer composition including the conjugated oligomer 4,7-bis[5-[7′-[6,6,12,12-tetrakis(2-methylbutyl)indenofluoren-2-yl]-9,9-bis(2-ethylhexyl)- 9′,9′-bis(2-methylbutyl)-7,2′-bifluoren-2-yl]-thien-2-yl]-2,1,3-benzothiadiazole (OF-2).

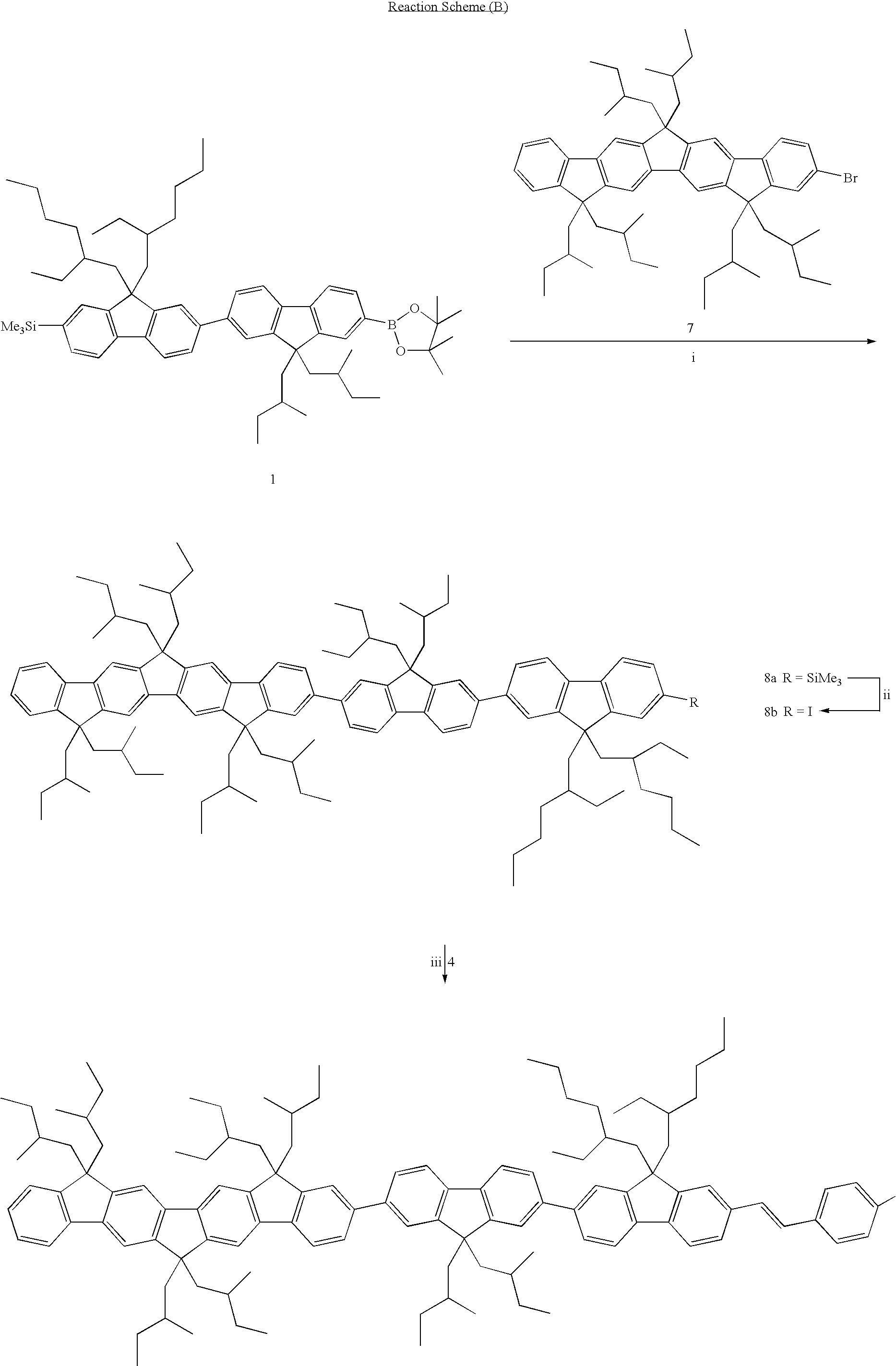

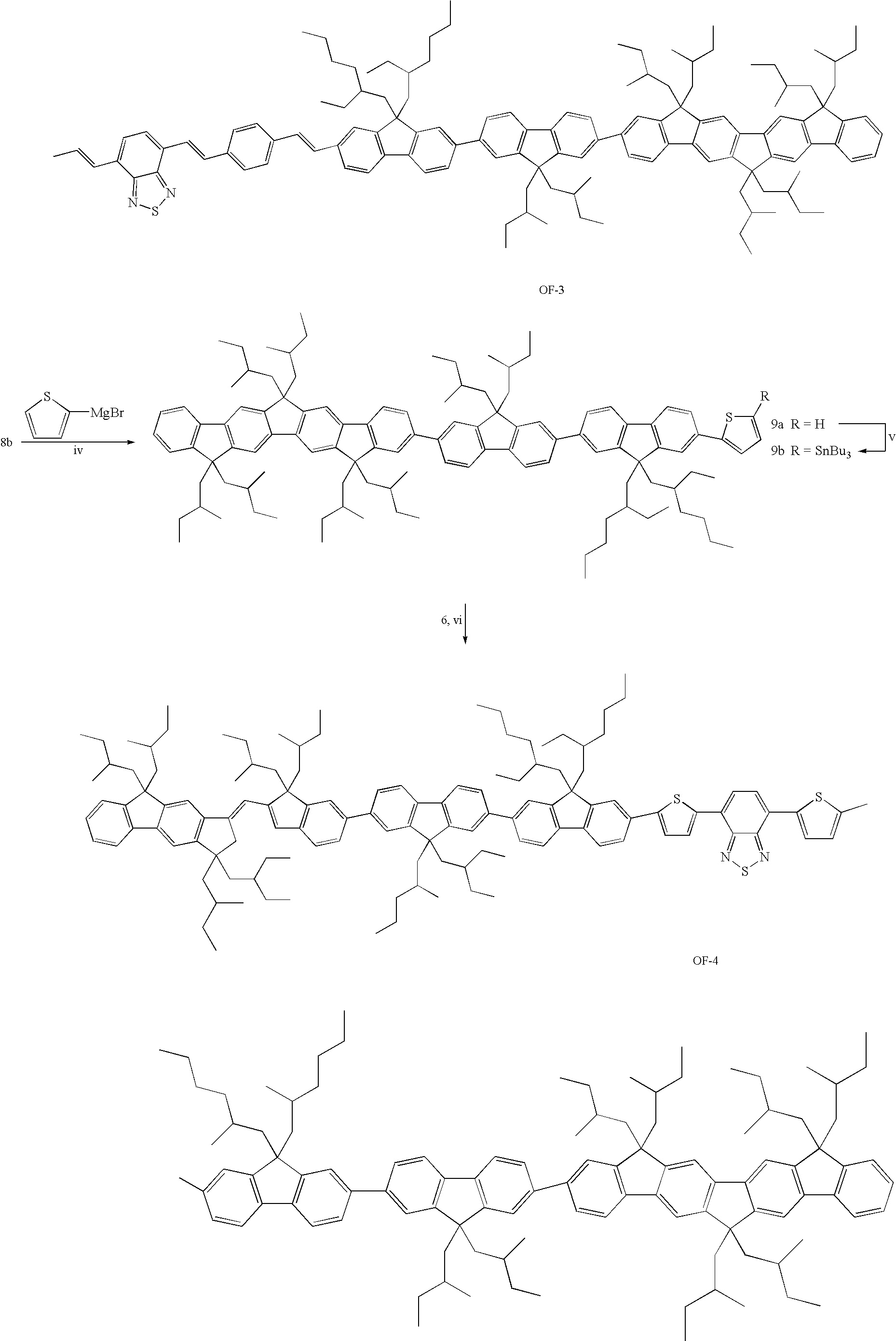

Reaction scheme (B) includes two synthetic pathways, through which monodisperse conjugated oligomer compositions according to this invention can be obtained. In particular, reaction scheme (B) provides the conjugated oligomers 4,7-bis[2-[4-[2-[4-[2-[7′-[6,6,12,12,15,15-hexakis(2-methylbutyl)bisindenofluoren-2-yl]-9,9-bis(2-ethylhexyl)-9′, 9′-bis(2-methylbutyl)2,2′-bifluoren-7-yl]ethenyl]phenyl]ethenyl]phenyl]ethenyl]-2,1,3-benzothiadiazole (OF-3) and 4,7-bis[5-[7′-[6,6,12,12,15,15-hexakis(2-methylbutyl)bisindenofluoren-2-yl]-9,9-bis(2-ethylhexyl)-9′,9′-bis(2-methylbutyl)-7,2′-bifluoren-2-yl]-thien-2-yl]-2,1,3-benzothiadiazole (OF-4).

According to the first synthetic pathway in reaction scheme (B), 2-[7′-trimethylsilyl-9,9-bis(2-methylbutyl)-9′, 9′-bis(2-ethylhexyl)-7,2′-bifluoren-2-yl]-4,4,5,5-tetramethyl-[1,3,2]dioxaborolane (1) is reacted with 2-bromo-6,6,12,12,15,15-hexakis(2-methylbutyl)bisindenofluorene (7) in the presence of Pd(PPh₃)₄ in a 2.0 M aqueous solution of Na₂CO₃ at a temperature of 90° C. to obtain 7-[6,6,12,12,15,15-hexakis(2-methylbutyl)bisindenofluoren-2-yl]-7′-trimethylsilyl-9,9-bis(2-methylbutyl)-9′,9′-bis(2-ethylhexyl)-2,2′-bifluorene (8a).

The obtained 7-[6,6,12,12,15,15-hexakis(2-methylbutyl)bisindenofluoren-2-yl]-7′-trimethylsilyl-9,9-bis(2-methylbutyl)-9′, 9′-bis(2-ethylhexyl)-2,2′-bifluorene (8a) is reacted with IC1 at 0° C. to obtain 7-iodo-7′-[6,6,12,12,15,15-hexakis(2-methylbutyl)bisindenofluoren-2-yl]-9,9-bis(2-ethylhexyl)- 9′,9′-bis(2-methylbutyl)-2,2′-bifluorene (8b).

The obtained 7-iodo-7′-[6,6,12,12,15,15-hexakis(2-methylbutyl)bisindenofluoren-2-yl]-9,9-bis(2-ethylhexyl)-9′,9′-bis(2-methylbutyl)-2,2′-bifluorene (8b) is reacted with 4,7-bis(p-vinylstyryl)-2,1,3-benzothiadiazole (4) in the presence of Pd(OAc)₂, K₂CO₃ and Bu₄NBr at 110° C. to obtain a monodisperse oligomer composition including the conjugated oligomer 4,7-bis[2-[4-[2-[4-[2-[7′-[6,6,12,12,15,15-hexakis(2-methylbutyl)bisindenofluoren-2-yl]-9,9-bis(2-ethylhexyl)-9′,9′-bis(2-methylbutyl)-2,2′-bifluoren-7-yl]ethenyl]phenyl]ethenyl]phenyl]ethenyl]-2,1,3-benzothiadiazole (OF-3).

According to the second synthetic pathway in reaction scheme (B), 7-iodo-7′-[6,6,12,12,15,15-hexakis(2-methylbutyl)bisindenofluoren-2-yl]-9,9-bis(2-ethylhexyl)-9′,9′-bis(2-methylbutyl)2,2′-bifluorene (8b) is reacted with 2-thienylmagnesium bromide in the presence of Pd(dppf)Cl₂ at room temperature to obtain 2-[7′-[6,6,12,12,15,15-hexakis(2-methylbutyl)bisindenofluoren-2-yl]-9,9-bis(2-ethylhexyl)- 9′,9′-bis(2-methylbutyl)7,2′-bifluoren-2-yl]-thiophene (9a).

The obtained 2-[7′-[6,6,12,12,15,15-hexakis(2-methylbutyl)bisindenofluoren-2-yl]-9,9-bis(2-ethylhexyl)-9′,9′-bis(2-methylbutyl)7,2′-bifluoren-2-yl]-thiophene (9a) is reacted with n-BuLi at 0° C. and then Bu₃SnCl at −78° C. The mixture is then returned to room temperature. 2-[7′-[6,6,12,12,15,15-Hexakis(2-methylbutyl)bisindenofluoren-2-yl]-9,9-bis(2-ethylhexyl)- 9′,9′-bis(2-methylbutyl)-7,2′-bifluoren-2-yl]-5-tributylstanyl-thiophene (9b) is thus obtained.

The obtained 2-[7′-[6,6,12,12,15,15-hexakis(2-methylbutyl)bisindenofluoren-2-yl]-9,9-bis(2-ethylhexyl)-9′,9′-bis(2-methylbutyl)-7,2′-bifluoren-2-yl]-5-tributylstanyl-thiophene (9b) is reacted with 4, 7-dibromo-2,1,3-benzothiadiazole (6) in the presence of Pd(PPh₃)₄ at 100° C. to obtain a monodisperse oligomer composition including the conjugated oligomer 4,7-bis[5-[7′-[6,6,12,12,15,15-hexakis(2-methylbutyl)bisindenofluoren-2-yl]-9,9-bis(2-ethylhexyl)-9′,9′-bis(2-methylbutyl)-7,2′-bifluoren-2-yl]-thien-2-yl]-2,1,3-benzothiadiazole (OF-4).

While this invention has been described in conjunction with the exemplary embodiments outlined above, various alternatives, modifications, variations, improvements and/or substantial equivalents, whether known or that are or may be presently unforeseen, may become apparent to those having at least ordinary skill in the art. Accordingly, the exemplary embodiments of the invention, as set forth above, are intended to be illustrative, not limiting. Various changes may be made without departing from the spirit and scope of the invention. Therefore, the invention is intended to embrace all known or later developed alternatives, modifications, variations, improvements and/or substantial equivalents.

1. An oligomer composition comprising oligomers including fluorene residues, fused fluorene residues and aromatic groups.

2. The oligomer composition of claim 1, wherein the oligomers are oligomers given by formula (I): 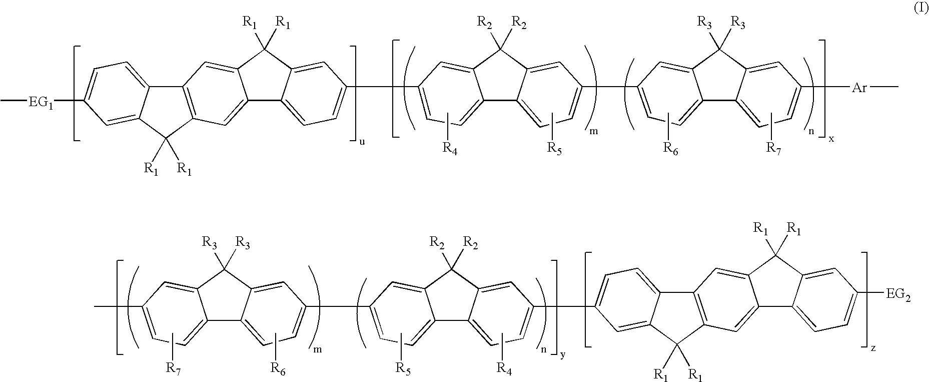
wherein:
R₁, R₂, R₃, R₄, R₅, R₆, R₇ each independently represent C_{q} branched or straight chain aliphatic groups, preferably C_{q}H_{2q+1} or (CH₂CH₂O)_{q}CH₃, racemic, chiral or achiral;
q represents an integer of from about 1 to about 40, preferably from 1 to 20;
m, n, u, x, y, z each independently represent an integer of from 0 to about 10, and u+z+(m+n)(x+y) is an integer of from about 2 to about 40, preferably from about 2 to about 20;
EG₁, EG₂ each independently represent end groups, preferably selected from the group consisting of H, a linear or branched alkyl group, and an unsubstituted or substituted aryl group; and
Ar represents a group selected from the group consisting of: 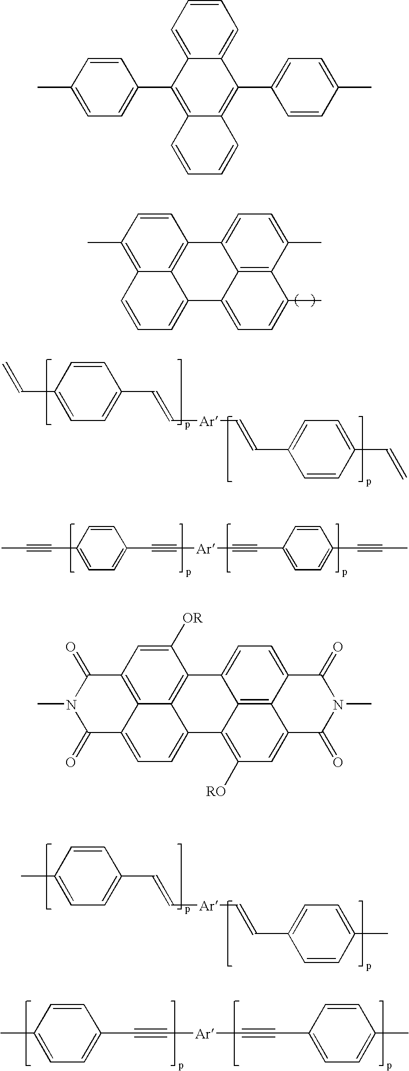
where p=0 to 10, preferably from 0 to 5; and
Ar′ represents a group selected from the group consisting of:

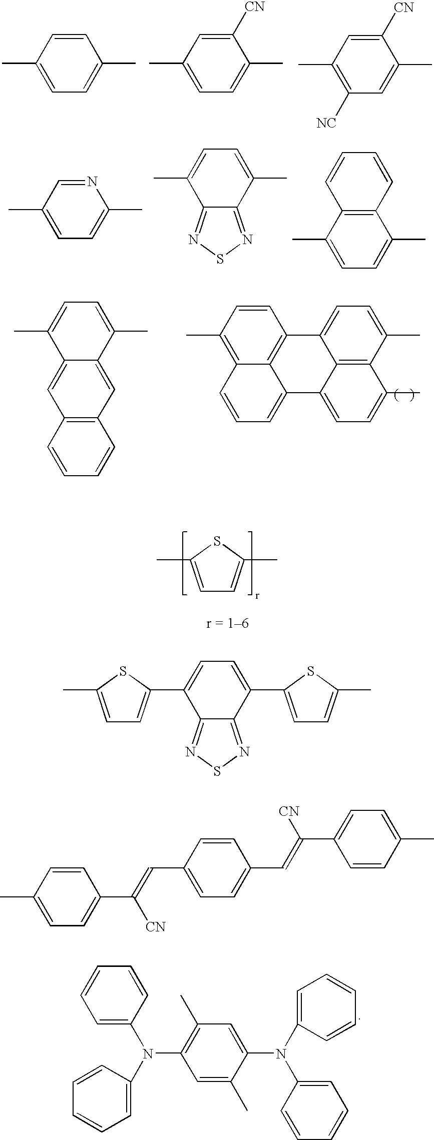

3. The oligomer composition of claim 1, wherein the oligomers are oligomers given by formula (II): 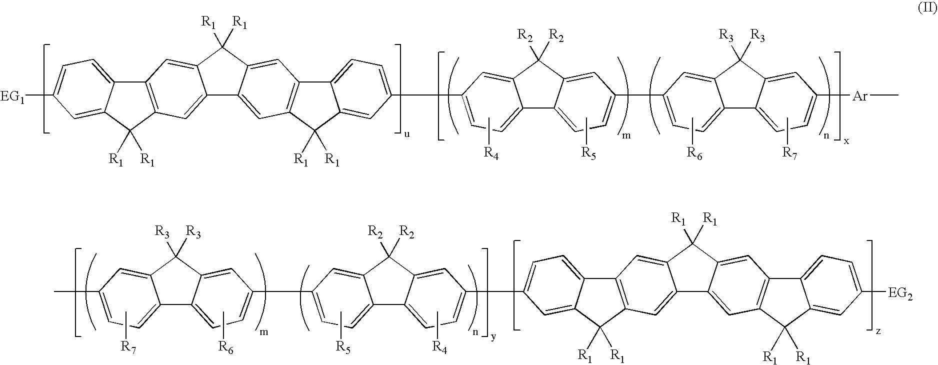
wherein:
R₁, R₂, R₃, R₄, R₅, R₆, R₇ each independently represent C_{q} branched or straight chain aliphatic groups, preferably C_{q}H_{2q+1} or (CH₂CH₂O)_{q}CH₃, racemic, chiral or achiral;
q represents an integer of from about 1 to about 40, preferably from 1 to 20;
m, n, u, x, y, z each independently represent an integer of from 0 to about 10, and u+z+(m+n)(x+y) is an integer of from about 2 to about 40, preferably from about 2 to about 20;
EG₁, EG₂ each independently represent end groups, preferably selected from the group consisting of H, a linear or branched alkyl group, and an unsubstituted or substituted aryl group; and
Ar represents a group selected from the group consisting of: 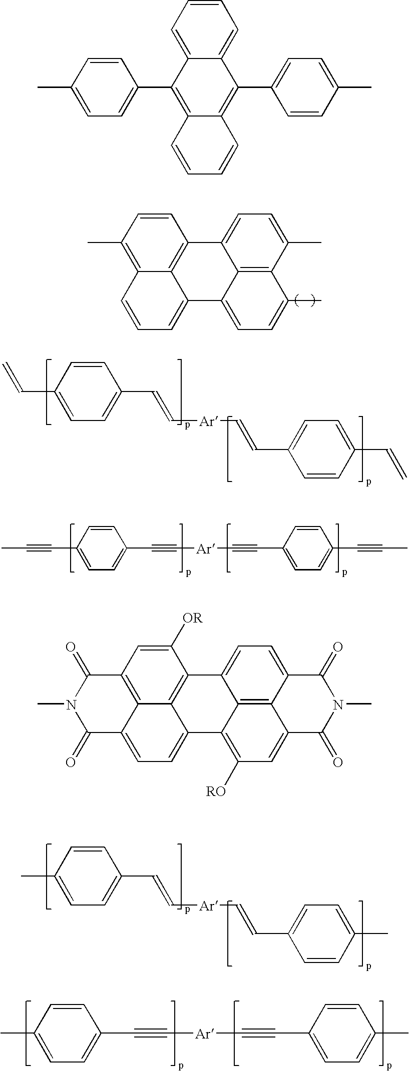
where p=0 to 10, preferably from 0 to 5; and
Ar′ represents a group selected from the group consisting of:

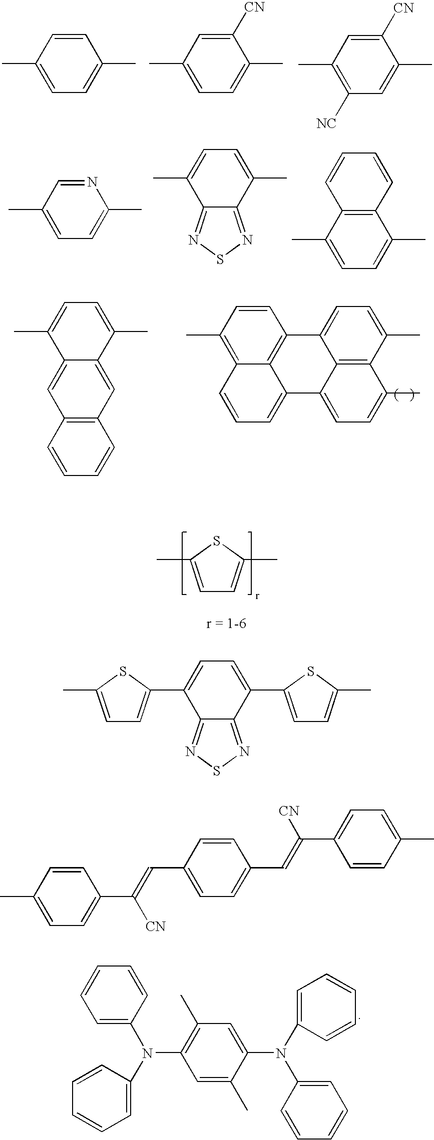

4. The oligomer composition of claim 1, wherein the composition has a polydispersity of about 1.

5. The oligomer composition of claim 1, wherein the composition is monodisperse.

6. The oligomer composition of claim 1, wherein the composition displays nematic mesomorphism.

7. The oligomer composition of claim 1, wherein the composition has a glass transition temperature of from about 90° C. to about 160° C.

8. The oligomer composition of claim 1, wherein the composition has a nematic-to-isotropic transition temperature of at least about 300° C.

9. The oligomer composition of claim 1, wherein the composition has a nematic-to-isotropic transition temperature of at least about 350° C.

10. A glassy nematic film, comprising the composition of claim 1.

11. An organic light-emitting device, comprising the composition of claim 1.

12. A glassy nematic film, comprising a film formed by depositing a composition comprising oligomers including fluorene residues, fused fluorene residues and aromatic groups on a substrate.

13. The glassy nematic film of claim 12, wherein the composition is deposited by spin-coating.

14. The glassy nematic film of claim 12, wherein the film has an orientational order parameter of at least about 0.70.

15. The glassy nematic film of claim 12, wherein the film has an emission dichroic ratio of at least about 8.0.

16. The glassy nematic film of claim 12, wherein the electron transition dipoles of central segments of the oligomers are substantially parallel to molecular long axes of the oligomers.

17. An organic light-emitting device, comprising the glassy nematic film of claim 12.

18. The organic light emitting device of claim 17, wherein the device emits light with a peak polarization ratio of at least about 12.

19. The organic light emitting device of claim 17, wherein the device emits light with a luminance yield of at least about 0.40 cd/A at a current of about 20 mA/cm².

20. A method for preparing an oligomer composition, comprising:
obtaining fluorene residues;
obtaining fused fluorene residues;
reacting the fluorene residues with the fused fluorene residues to obtain combined fluorene/fused fluorene residues; and
reacting the combined fluorene/fused fluorene residues with an aromatic compound to obtain conjugated oligomers.

21. The method of claim 20, wherein obtaining fluorene residues comprises obtaining fluorene residues including two or more fluorenes.

22. The method of claim 20, wherein obtaining fluorene residues comprises obtaining fluorene residues functionalized with dioxaborolane and trimethylsilyl groups.

23. The method of claim 20, wherein obtaining fused fluorene residues comprises obtaining fused fluorene residues including three or more six-carbon rings.

24. The method of claim 20, wherein obtaining fused fluorene residues comprises obtaining fused fluorene residues functionalized with one or more halogen groups.

25. The method of claim 24, wherein obtaining fused fluorene residues comprises obtaining fused fluorene residues functionalized with one or more bromine groups.

26. The method of claim 20, wherein reacting the fluorene residues with the fused fluorene residues to obtain combined fluorene/fused fluorene residues comprises reacting the fluorene residues with the fused fluorene residues in the presence of a catalyst.

27. The method of claim 26, wherein reacting the fluorene residues with the fused fluorene residues to obtain combined fluorene/fused fluorene residues comprises reacting the fluorene residues with the fused fluorene residues in the presence of a palladium catalyst.

28. The method of claim 20, wherein reacting the combined fluorene/fused fluorene residues with an aromatic compound to obtain conjugated oligomers comprises substituting functional groups of the combined fluorene/fused fluorene residues.

29. The method of claim 28, wherein substituting functional groups of the combined fluorene/fused fluorene residues comprises replacing trimethylsilyl groups with halogen groups.

30. The method of claim 29, wherein substituting functional groups of the combined fluorene/fused fluorene residues comprises replacing trimethylsilyl groups with iodine groups.

31. The method of claim 20, wherein reacting the combined fluorene/fused fluorene residues with an aromatic compound to obtain conjugated oligomers comprises:
reacting the combined fluorene/fused fluorene residues with a first aromatic compound to obtain aromatic intermediates; and
reacting the aromatic intermediates with a second aromatic compound to obtain conjugated oligomers.

32. The method of claim 20, wherein reacting the combined fluorene/fused fluorene residues with an aromatic compound to obtain conjugated oligomers comprises reacting the combined fluorene/fused fluorene residues with an aromatic compound in the presence of a catalyst.

33. The method of claim 32, wherein reacting the combined fluorene/fused fluorene residues with an aromatic compound to obtain conjugated oligomers comprises reacting the combined fluorene/fused fluorene residues with an aromatic compound in the presence of a palladium catalyst.
